(19) United States
(12) Patent Application Publication
(10) Pub. No.: US 2009/0118331 A1
(43) Pub. Date: May 7, 2009

(54) Use of Lobeline Epimers in the Treatment of Central Nervous System Diseases, Pathologies, and Drug Abuse

(76) Inventors: Peter A. Crooks, Nicholasville, KY (US); Linda P. Dwoskin, Lexington, KY (US)

Correspondence Address: GFD PATENTS, LLC, P.O. BOX 752, CLIFTON PARK, NY, 12065, US

(21) Appl. No.: 12/263,458

(22) Filed: Nov. 1, 2008

Related U.S. Application Data
(60) Provisional application No. 60/985,189, filed on Nov. 2, 2007.

(51) Int. Cl. A61K 31/445; A61P 25/00

(52) U.S. Cl. 514/317

(57) ABSTRACT

Methods of delivering or administering stabilized formulations or compositions having predetermined ratios, or range of ratios, of constituent epimers to an individual or a mammal for treatment of central nervous system diseases, pathologies, and drug abuse and compositions for stabilizing the compositions. In one embodiment, the predetermined ratios of constituent epimers, or range of ratios, are predetermined ratios of 2-[6S-(2S-hydroxy-2-phenyl-ethyl)-1-methyl-piperidin-2R-yl]-1-phenyl-ethanone (2R-lobeline) and its epimer, 2-[6S-(2S-hydroxy-2-phenyl-ethyl)-1-methyl-piperidin-2S-yl]-1-phenyl-ethanone (2S-lobeline). In embodiments, the stabilized formulations or compositions of 2R- and 2S-lobeline are provided in the ranges between 1 part 2R-lobeline to 10000 parts 2S-lobeline to 10000 parts 2R-lobeline to 1 part 2S-lobeline, or in the range of a 1 to 1 mixture of 2R- and 2S-lobeline, so that the predetermined epimeric ratio of 2R- and 2S-lobeline is delivered or administered to the blood, plasma or tissues of a patient so treated.

The present patent application is a non-provisional application claiming priority from provisional application Ser. No.: 60/985,189 (filed Nov. 2, 2007 and entitled “Use of Lobeline Diastereomers in the Treatment of Central Nervous System Diseases, Pathologies, and Drug Abuse”).

### BACKGROUND

1. Field of the Invention

The present invention relates generally to stabilized formulations or compositions having predetermined ratios, or a range of ratios, of constituent epimers, to an individual or a mammal for treatment of central nervous system diseases, pathologies, and drug abuse and compositions for stabilizing the compositions, and methods of delivering or administering the same. More specifically, the stabilized formulations or compositions having predetermined ratios, or a range of ratios of the present invention relate to stabilized formulations or compositions having predetermined ratios, or a range of ratios, of 2R- and 2S-lobeline epimers (“2R/2S-lobeline”), and methods for delivering or administering the stabilized formulations or compositions to achieve a certain plasma, blood or tissue ratio, or range of ratios, of 2R- and 2S-lobeline epimers in vivo in an individual or mammal, which are effective for the treatment of diseases and pathologies of the central nervous system (CNS), or for the treatment of drug abuse and withdrawal therefrom.

2. Related Art

A method of treating an individual having a central nervous system disease or pathology has been disclosed by the present inventors in U.S. Pat. No. 6,087,376 (Jul. 11, 2000) to Crooks and Dwoskin, and is herein incorporated by reference. In U.S. Pat. No. 6,087,376, the central nervous system disease or pathology is selected from the group consisting of head or brain trauma, psychosis, sleep disorders, obsessive-compulsive disorders, panic disorders, myasthenia gravis, Parkinson's disease, schizophrenia, Tourette's syndrome, Huntington's disease, and attention deficit disorder. A method of treating an individual for dependence on a drug of abuse, withdrawal from a drug of abuse, or for an eating disorder has been disclosed by the present inventors in U.S. Pat. No. 5,830,904 (Nov. 3, 1998) to Crooks and Dwoskin, and is herein incorporated by reference. In U.S. Pat. No. 5,830,904 the drug of abuse is selected from the group consisting of cocaine, amphetamines, caffeine, phencyclidine, opiates, barbiturates, benzodiazepines, cannabinoids, hallucinogens and alcohol.

There is a need for improved treatments of central nervous system diseases and pathologies and drug abuse.

### SUMMARY OF THE INVENTION

A first aspect of the present invention provides a method of treating a patient or mammal for a central nervous system disease or pathology, or for dependence on a drug of abuse or withdrawal from a drug of abuse, or for reducing an individual's desire for food, by delivering to an individual an effective amount of an epimeric mixture of 2-[6S-(2S-hydroxy-2-phenyl-ethyl)-1-methyl-piperidin-2R-yl]-1-phenyl-ethanone (2R-lobeline) and its epimer, 2-[6S-(2S-hydroxy-2-phenyl-ethyl)-1-methyl-piperidin-2S-yl]-1-phenyl-ethanone (2S-lobeline), where 2R-lobeline and 2S-lobeline are represented by the following structural formulas:

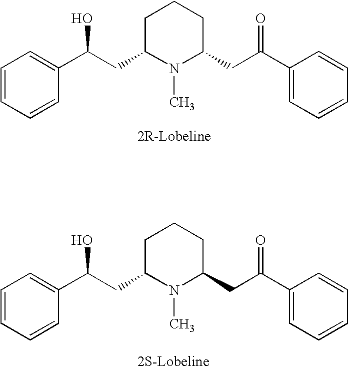

A second aspect of the present invention provides an in vivo stabilized 2R- and 2S-Lobeline composition in blood, plasma, tissue or cytosol, comprising: a predetermined 2R- and 2S-lobeline mixture ranging between 1 part 2R-lobeline to 10000 parts 2S-lobeline and 10000 parts 2R-lobeline to 1 part 2S-lobeline, or a 1 to 1 mixture of 2R- and 2S-lobeline, wherein 2R-lobeline and 2S-lobeline have the following structural formulas:

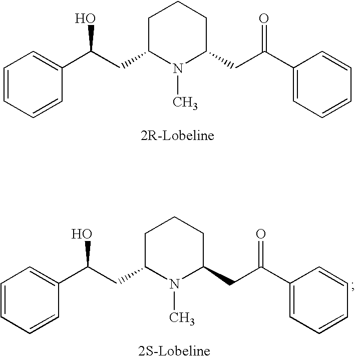

and a solvent or one or more pharmaceutical excipients.

A third aspect of the present invention provides a method for delivering to a patient in need thereof a stabilized epimeric mixture of 2R- and 2S-lobeline having a predetermined epimeric ratio, comprising: providing an epimeric mixture having a ratio of 2R- and 2S-lobeline ranging between 1 part 2R-lobeline to 10000 parts 2S-lobeline and 10000 parts 2R-lobeline to 1 part 2S-lobeline, or a 1 to 1 mixture of 2R- and 2S-lobeline, so that it is effective for delivering to the patient in need thereof the predetermined epimeric mixture to the blood, plasma or tissues of the patient and a solvent or one or more pharmaceutical excipients.

### BRIEF DESCRIPTION OF THE DRAWINGS

FIG. 1 depicts plasma concentration-time curves for 4 of the subjects, in accordance with embodiments of the present invention.

### DESCRIPTION OF THE INVENTION

The use of lobeline compounds in the treatment of central nervous system diseases and pathologies has been described and disclosed in previous patents by the authors of the present invention, which are incorporated herein in their entirety by reference (Crooks and Dwoskin, U.S. Pat. No. 5,830,904, 1998; Crooks and Dwoskin, U.S. Pat. No. 6,087,376, 2000). Hereinafter, “lobeline” refers to 2R-lobeline and “epimer” or “it's epimer” refers to 2S-lobeline. Hereinafter “lobeline epimer” means 2S-lobeline.

These previous patents relate to the use of lobeline and lobeline analogs for the palliation or treatment of certain pathologies and conditions. However, the actual ratio of lobeline epimers (“epimeric mixture”) that may be present in the pharmacological assay media employed in the in vitro assays used to delineate the mechanisms underlying the pharmacological effects of lobeline reported in these patents, and the rate and extent of lobeline epimerization in these media, have not been described. Likewise, the epimeric mixture or mixtures that may be present in vivo after lobeline administration to a mammal or an individual that are responsible for its pharmacological activity in a mammal or the individual, have never been reported.

Thus, in vitro and in vivo epimerization and pharmacokinetic studies were performed as reported herein in order to characterize the tendency of lobeline to undergo epimerization, and to assess the rate, extent and ratio of lobeline epimerization under various conditions. These studies were performed in order to better appreciate the likely range of lobeline to lobeline epimer ratio or ratios (epimeric mixtures) that have pharmacological relevance, both in vitro and in vivo.

Based upon the extensive in vitro lobeline epimerization studies summarized herein, it is evident that lobeline epimerization is highly dependent upon the characteristics of the dissolution medium, including pH and perhaps the presence of other solutes. The rate and extent of lobeline epimerization and, consequently, the ratio of lobeline to lobeline epimer (epimeric mixture) present in solution, is dynamic and variable with time. This may be particularly important in the context of lobeline pharmacological assays, since it is clear that lobeline is present as an epimeric mixture in aqueous assay media, and it may be one particular epimeric mixture, or a discrete range of epimeric mixtures, that is actually responsible for observed pharmacological effects in vitro.

The studies described herein also reveal that lobeline undergoes extensive epimerization when administered in vivo, giving rise to an epimeric mixture in the plasma. In rats, this epimeric mixture appears to vary between male and female animals. The relative epimeric ratio or mixture observed in rat plasma also varies depending upon the route of administration (here, intravenous, subcutaneous and oral), employed to deliver an epimeric mixture to the animal. In rats, the overall ratio of lobeline to epimer in the epimeric mixture detected in the plasma, as represented by a comparison of relative areas under the curve (AUC), is approximately 1.5-2 to 1, depending upon both the sex of the animal and the route of administration. In contrast, the ratio of epimer to lobeline detected in the plasma of humans receiving sublingual lobeline is on the order of approximately 8-10 to 1, although this ratio varies somewhat between individuals, and is not immediately predictable. Moreover, although the overall range of epimer to lobeline ratios in the epimeric mixture detected in the plasma may be relatively narrow, the plasma concentration-time curves of lobeline and lobeline epimer still vary between individuals in an unpredictable manner. In addition, this ratio may be influenced by the dose administered to an individual.

Accordingly, one aspect of the present invention relates to delivering to a mammal or an individual an amount of an epimeric mixture of 2-[6S-(2S-hydroxy-2-phenyl-ethyl)-1-methyl-piperidin-2R-yl]-1-phenyl-ethanone (2R-lobeline) and its epimer, 2-[6S-(2S-hydroxy-2-phenyl-ethyl)-1-methyl-piperidin-2S-yl]-1-phenyl-ethanone (2S-lobeline) effective for the palliation or treatment of a disease or pathology selected from the group consisting of head or brain trauma, pain management, psychosis, affective disorders, personality disorders, dyssomnias, including narcolepsy and other sleep disorders, eating disorders including obesity, obsessive-compulsive disorders, panic disorders, schizophrenia, myasthenia gravis, Parkinson's disease, hyperkinetic disorders, Tourette's syndrome, Huntington's disease, and attention deficit hyperactivity and conduct disorders, disorders of learning and memory; and drug abuse, wherein said drug of abuse is selected from the group consisting of cocaine, amphetamines, caffeine, phencyclidine, opiates, barbiturates, benzodiazepines, cannabinoids, hallucinogens, inhalants, psychedelics, and alcohol.

The studies summarized herein reveal that 2R-lobeline, when administered sublingually, undergoes epimerization in vivo to form epimeric mixtures in a dynamic and not necessarily predictable way. The administration of an epimeric mixture of lobeline may also epimerize, and may also be expected to elicit desirable pharmacological effects. Thus, in another embodiment, the present invention relates to the use or delivery of an epimeric mixture of lobeline, wherein the epimeric mixture comprises a mixture of 2R-lobeline and 2S-lobeline in any epimeric ratio of 2R- and 2S-lobeline, ranging between 1 part 2R-lobeline to 10000 parts 2S-lobeline and 10000 parts 2R-lobeline to 1 part 2S-lobeline, or is a 1 to 1 mixture of 2R- to 2S-lobeline.

In one embodiment of the present invention, this epimeric mixture may contain the free base forms of the lobeline epimers, and their polymorphs.

In another embodiment of the invention, the epimeric mixture may contain one or more pharmaceutically acceptable salts of the epimers, wherein the salt is selected from the group consisting of acetate, benzenesulfonate, benzoate, bicarbonate, bromide, calcium edetate, camphorsulfonate, carbonate, hydrochloride, citrate, edetate, fumarate, glucoheptonate, gluconate, glutamate, hyclate, hydrobromide, hydrochloride, hydroiodide, lactate, lactobionate, lauryl sulfonate, malate, maleate, mandelate, methanesulfonate, mucate, nitrate, pamoate, pantothenate, phosphate, polygalacturonate, salicyclate, sodium succinate, stearate, subacetate, succinate, sulfate, tosylate, tannate, tartrate, adipate, alginate, aspartate, bisulfate, hemisulfate, hydrofluoride, oxalate, pectinate, persulfate, propionate, undecanoate, adipoate, arginate, aspartate, betaine, carnitine, decanoate, dioctylsulfosuccinate, pamoate, fructose-1,6-diphosphate, glucose phosphate, L-glutaminate, lauryl sulfate, lysine, octanonate, tannate, and combinations thereof, and their polymorphs.

In another embodiment of the present invention, the epimeric mixture may contain a solvate, complex or dispersion of lobeline epimers combined with a pharmaceutically acceptable solvent, wherein the solvent is selected from the group of solvents consisting of water, saline, aqueous buffers, acetone, ethanol, methanol, isopropanol, isobutanol, tertiary butanol, ethyl acetate, methylene chloride, acetonitrile, glycerin, propylene glycol, liquid paraffin, mineral oil, ethylene glycol, butanol, ethoxyethanol, ethyl ether, isobutyl acetate, isopropyl acetate, propanol, chloroform, butyl acetate, diethylene glycol monoethyl ether, dimethyl sulfoxide, methane sulfonyl methane, and combinations thereof, and their polymorphs.

The epimeric mixture may be administered in a pharmaceutical formulation, or composition, comprising an epimeric mixture along with one or more pharmaceutical excipients. Generally, the composition or formulation would comprise an epimeric mixture along with pharmaceutical excipients selected from the non-limiting group consisting of solvents, such as ethanol and diethylene glycol monoethyl ether, surfactants, such as polysorbates, lecithin, fatty acid salts and alcohols, polymers, adhesives such as acrylates and polycarboxylates, binders, fillers and bulking agents, such as starch, lactose and mannitol, preservatives, such as tocopherol and BHT, and combinations thereof.

The studies summarized herein reveal that lobeline undergoes dynamic epimerization both in vitro and in vivo. Thus, the pharmacological mechanisms and effects ascribed to lobeline may be attributed to a mixture of lobeline epimers in solution and in vivo, such as in the plasma, blood or in target tissues or cytosol. Since the ratio of epimer to lobeline in such an epimeric mixture may change over time both in pharmacological assays and in vivo, there may be a specific epimer to lobeline ratio or epimeric mixture, or a range of ratios or epimeric mixtures, responsible for the pharmacological effects attributed to lobeline. Thus, it may be advantageous to administer or deliver to an individual or a mammal an epimeric mixture comprising a specific ratio or range of ratios of 2R-lobeline to 2S-lobeline, in order to rapidly and effectively achieve ideal blood, plasma, tissue and cytosol concentrations and epimeric mixtures; thus, pharmacological response.

Since a particular ratio of lobeline to epimer, or a range of lobeline to epimer ratios, may be advantageous for achieving a desired pharmacological effect, the use or delivery of lobeline in the form of lobeline and its epimer in a ratio that falls within a specified range of lobeline to epimer ratios, by pharmaceutical formulation or by otherwise stabilizing lobeline and its epimer in such a ratio or range of ratios through the use of appropriate chemicals, excipients and pharmaceutical formulation techniques, may be desirable. Accordingly, another aspect of the present invention is the formulation of an epimeric mixture such that said formulation contains an epimeric mixture, stabilized in any epimeric ratio, wherein said ratio of 2R- and 2S-lobeline ranges between 1 part 2R-lobeline to 10000 parts 2S-lobeline and 10000 parts 2R-lobeline to 1 part 2S-lobeline, or is a 1 to 1 mixture of 2R- and 2S-lobeline, and is effective for delivering an epimeric mixture to the blood, plasma or tissues of an individual or mammal so treated.

The rate and extent of epimerization of certain compounds has been moderated by limiting their hydration or exposure to humidity or bulk water. In the present invention, the stabilization of the epimeric ratio of lobeline and its epimer may be achieved by controlling the physical-chemical properties of a dispersion of the epimeric mixture. Said physical-chemical properties may include pH, temperature, composition, hydrogen bonding or electrostatic interactions, hydrophobic interactions, the degree of molecular ordering of the continuous or discontinuous phase, including the stabilization or disruption of molecular ordering through the use of chaotropic or kosmotropic agents, such as monovalent, divalent or polyvalent anions or cations, urea, guanidine, peptides and others, dielectric constant, ionic strength, pressure, and optical activity. The dispersion may be a solution, a solid solution, a suspension, a colloid, an adsorbed complex, a particle, a mass, a melt or fusate, a co-precipitation, a co-crystallization, a sublimate, a coacervation, a film, an extrusion, a compression, a physical mixture, and combinations thereof. Hereinafter “coacervation” means a reversible, emulsoid stage existing between sol and gel formations of the epimeric mixture, in which the addition of a third substance causes the separation of the sol into two immiscible liquid phases.

The dispersion may be formed by combining the epimeric mixture with one or more excipients. The excipients may include solvents, co-solvents, surfactants, co-surfactants, emulsifiers, and divided solids, such as colloidal particles, asymmetric or symmetric small molecules and their mixtures and racemates, including sugars, such as D- or L-glucose, amino acids, such as D- or L-lysine, other small molecules, such as D- or L-menthol, organic acids and bases, such as L-lactic acid, natural or synthetic polymers, both asymmetric and symmetric, such as oligo- and polysaccharides, oligo- and polypeptides, polyacrylates, polyvinylpyrrolidone, and polyethylene glycol, and other biomolecules, such as proteins, enzymes and membranes, and combinations thereof.

The method of combining may be physical mixing, including milling and blending, complexation, adsorption, coacervation, sublimation, co-precipitation, co-crystallization, desolvation, such as evaporation, spray drying, fluid extraction, supercritical fluid extraction, expansion of a supercritical fluid, and countercurrent exchange, fusion, such as melting, compression, extrusion, dissolution, emulsification, and combinations thereof.

Generally, the amount of epimeric mixture administered is effective to deliver an epimeric mixture in a pharmacologically active ratio or range of ratios, thus achieving a plasma epimer to lobeline ratio that is therapeutically effective, and thus to alleviate at least one of the symptoms of the specified conditions. As stated, the rate and extent of lobeline epimerization may vary by individual, species, route of administration, and perhaps dose in a non-obvious way. The pharmacological mechanisms and effects ascribed to lobeline may be attributed to a mixture of lobeline epimers in solution and in vivo in the plasma, blood or in target tissues or cytosol. In addition, since the ratio of epimer to lobeline in such an epimeric mixture may change over time both in pharmacological assays and in vivo, it is evident that there may be a specific epimer to lobeline ratio or epimeric mixture, or a range of ratios or epimeric mixtures, responsible for the pharmacological effects attributed to lobeline. As summarized herein, the equilibrium ratio of lobeline to epimer in rats is approximately 1.5-2 to 1. In contrast, it is approximately 1 to 10 in humans. Accordingly, one aspect of the present invention relates to the use and administration of lobeline epimers in specific ratios for the delivery of epimeric mixtures to a mammal and the ultimate delivery of an epimeric mixture to the plasma, blood, target tissues, receptors and cytosol of an individual or a mammal, for the palliation and treatment of the aforementioned conditions. Thus, another aspect of the present invention relates to the administration or delivery of an epimeric mixture to an individual or a mammal to produce a mixture of 2R-lobeline and 2S-lobeline epimers in the blood, plasma or tissue of an individual, wherein the lobeline mixture, (2R/2S-lobeline), delivered systemically, comprises a mixture of 2R-lobeline and 2S-lobeline in any epimeric ratio of 2R- and 2S-lobeline, ranging between 1 part 2R-lobeline to 10000 parts 2S-lobeline and 10000 parts 2R-lobeline to 1 part 2S-lobeline, or is a 1 to 1 mixture of 2R- to 2S-lobeline.

Another aspect of the present invention relates to the administration or delivery of an epimeric mixture to an individual or a mammal to produce a mixture of 2R-lobeline and 2S-lobeline epimers in the blood, plasma or tissue of an individual, wherein said blood, plasma or tissue ratio of 2R- and 2S-lobeline ranges between 1 part 2R-lobeline to 100 parts 2S-lobeline and 100 parts 2R-lobeline to 1 part 2S-lobeline.

Another aspect of the present invention relates to the administration or delivery of an epimeric mixture to an individual or a mammal to produce a mixture of 2R-lobeline and 2S-lobeline epimers in the blood, plasma or tissue of an individual, wherein the delivery of an epimeric mixture produces a mixture of 2R-lobeline and 2S-lobeline epimers in the blood, plasma or tissue of an individual, wherein said blood, plasma or tissue ratio of 2R- and 2S-lobeline ranges between 1 part 2R-lobeline to 30 parts 2S-lobeline and 30 parts 2R-lobeline to 1 part 2S-lobeline.

Another aspect of the present invention relates to the administration or delivery of an epimeric mixture to an individual or a mammal to produce a mixture of 2R-lobeline and 2S-lobeline epimers in the blood, plasma or tissue of an individual, wherein the delivery of an epimeric mixture produces a mixture of 2R-lobeline and 2S-lobeline epimers in the blood, plasma or tissue of an individual, wherein the delivery of an epimeric mixture produces a mixture of 2R-lobeline and 2S-lobeline epimers in the blood, plasma or tissue of an individual, wherein said blood, plasma or tissue ratio of 2R- and 2S-lobeline is 1 part 2R-lobeline to 10 parts 2S-lobeline.

In an embodiment, the delivery of an epimeric mixture is achieved by administration subcutaneously, intramuscularly, intravenously, intrathecally, transdermally, orally, intranasally, sublingually, buccally, by inhalation or insufflation, by implantation, vaginally or rectally.

In an embodiment, the delivery of an epimeric mixture produces a mixture of 2R-lobeline and 2S-lobeline epimers in the blood, plasma or tissue of an individual or mammal, wherein said blood, plasma or tissue ratio of 2R- and 2S-lobeline ranges between 1 part 2R-lobeline to 30 parts 2S-lobeline and 30 parts 2R-lobeline to 1 part 2S-lobeline.

In an embodiment, the delivery of an epimeric mixture produces a mixture of 2R-lobeline and 2S-lobeline epimers in the blood, plasma or tissue of an individual or mammal, wherein said blood, plasma or tissue ratio of 2R- and 2S-lobeline ranges between 1 part 2R-lobeline to 100 parts 2S-lobeline and 100 parts 2R-lobeline to 1 part 2S-lobeline.

In an embodiment, the delivery of an epimeric mixture produces a mixture of 2R-lobeline and 2S-lobeline epimers in the blood, plasma or tissue of an individual or mammal, wherein said blood, plasma or tissue ratio of 2R- and 2S-lobeline is 1 part 2R-lobeline to 10 parts 2S-lobeline.

In an embodiment, the central nervous system disease or pathology is selected from the group consisting of head or brain trauma, pain management, psychosis, affective disorders, personality disorders, dyssomnias, including narcolepsy and other sleep disorders, eating disorders including obesity, obsessive-compulsive disorders, panic disorders, schizophrenia, myasthenia gravis, Parkinson's disease, hyperkinetic disorders, Tourette's syndrome, Huntington's disease, and attention deficit hyperactivity and conduct disorders, disorders of learning and memory; and drug abuse, wherein said drug of abuse is selected from the group consisting of cocaine, amphetamines, caffeine, phencyclidine, opiates, barbiturates, benzodiazepines, cannabinoids, hallucinogens, inhalants, psychedelics, and alcohol.

In one embodiment, a formulation or composition having a predetermined epimeric ratio for delivering or administering the predetermined epimeric ratio may comprise a predetermined epimeric mixture of 2R-lobeline and 2S-lobeline epimers and excipients.

In an embodiment, the epimeric mixture may comprise a mixture of 2R-lobeline and 2S-lobeline in any epimeric ratio of 2R- and 2S-lobeline, ranging between 1 part 2R-lobeline to 10000 parts 2S-lobeline and 10000 parts 2R-lobeline to 1 part 2S-lobeline, or is a 1 to 1 mixture of 2R- to 2S-lobeline.

In an embodiment, the epimeric mixture may be employed as the free base, or as pharmaceutically acceptable salts, solvates, complexes, dispersions or polymorphs thereof.

In an embodiment, the epimeric mixture may be administered in a pharmaceutical formulation, or composition, comprising an epimeric mixture along with one or more pharmaceutical excipients.

In an embodiment, the stabilized formulation or composition having a predetermined epimeric ratio may comprise an epimeric mixture having a ratio of 2R- and 2S-lobeline ranging between 1 part 2R-lobeline to 10000 parts 2S-lobeline and 10000 parts 2R-lobeline to 1 part 2S-lobeline, or is a 1 to 1 mixture of 2R- and 2S-lobeline, and is effective for delivering the respective epimeric mixture to the blood, plasma or tissues of an individual so treated.

Additional advantages of the present invention may become apparent to one skilled in the art.

The invention will now be discussed by way of certain examples, which illustrate, but in no way limit, the invention.

### EXAMPLES

### Lobeline Epimerization in Aqueous Media.

In order to appreciate the epimerization of lobeline in deionized water (Table 1) and in the HEPES buffers used in the [³H]DTBZ and VMAT2 uptake assays employed in previously reported (Crooks and Dwoskin, U.S. Pat. No. 6,087,376, 2000) in vitro lobeline pharmacological studies, an extensive evaluation of lobeline epimerization in HEPES buffer at 25° C. and 37° C. was conducted (Tables 2 and 3, respectively). The initial dissolution of lobeline in water was followed by a 1:1 dilution of this stock solution in the appropriate HEPES buffer system, incubation at the prescribed temperature, and HPLC assay of each epimer and potential degradants over 2 hour incubation. After 2 hours at 25° C., the final component concentration (as a function of the area under the curve) was 83% lobeline, and 17% epimer. A 2 hour incubation at 37° C. gave rise to 69% lobeline, 30% corresponding epimer, and ca. 1.6% impurities. In contrast, when lobeline is dissolved deionized (DI) in water, the change in epimer concentration was minimal (<1%) over a 2-h period at 25° C. This is consistent with previous observations that showed significant epimerization in DI water only after 24 or more hours. These studies clearly demonstrate the buffer-, time- and temperature-, and perhaps solute-dependent epimerization of lobeline in aqueous systems, including the HEPES buffers employed in the binding and uptake assays revealed herein.

Extensive in vitro studies on the epimerization of lobeline were conducted in a variety of aqueous media. As shown in Table 1, whether dissolved in fresh deionized water, or in water allowed to absorb atmospheric carbon dioxide for up to 8 hours, the conversion of lobeline to lobeline epimer was on the order of ca. 1 percent over the 2 hour time course of the experiment. The pH of the lobeline solutions employed in these studies was near neutral, suggesting that rapid epimeric conversion in water alone is minimal, and that if it is to occur, it is more likely to occur in the presence of a stronger buffer system, non-neutral pH, or in the presence of other solution components. The concentration of the lobeline solution (500 mL) was ca. 0.5 mg/mL, and 1.5 mL aliquot parts were removed in triplicate at each time point and analyzed by HPLC. Corresponding pH measurements were performed with a Coming Model 240 pH meter.

TABLE 1
Lobeline conversion to epimer in deionized water.

| | | | % Lobeline | % Epimer |
|---|---|---|---|---|
| Study | Time Point^{a} | pH | (Mean ± SD) | (Mean ± SD) |
| Solution 1 | 0 | 6.02 | 99.16 | 0.83 |
| (Lobeline in | 15 min | 6.03-6.24 | 99.17 ± 0.053 | 0.83 ± 0.054 |
| “fresh” | 30 min | 6.02-6.24 | 99.01 ± 0.162 | 0.99 ± 0.164 |
| DI Water) | 45 min | 6.13-6.37 | 99.06 ± 0.146 | 0.95 ± 0.144 |
| | 1 h | 6.26-6.52 | 99.00 ± 0.195 | 1.00 ± 0.196 |
| | 80 min | 6.19-6.50 | 98.54 ± 0.112 | 1.46 ± 0.112 |
| | 100 min | 6.16-6.50 | 98.50 ± 0.139 | 1.50 ± 0.139 |
| | 2 h | 5.63-5.88 | 98.42 ± 0.601 | 1.58 ± 0.599 |
| Solution 2 | 0 | 7.87 | 98.97 | 1.03 |
| (Lobeline | 15 min | 7.78-8.21 | 99.34 ± 0.274 | 0.66 ± 0.273 |
| in “8-h” | 30 min | 7.58-7.85 | 99.49 ± 0.215 | 0.52 ± 0.214 |
| DI Water) | 45 min | 7.66-7.68 | 99.35 ± 0.056 | 0.65 ± 0.056 |
| | 1 h | 6.84-7.11 | 99.20 ± 0.085 | 0.80 ± 0.087 |
| | 80 min | 6.53-6.79 | 99.11 ± 0.150 | 0.90 ± 0.150 |
| | 100 min | 6.24-6.51 | 98.99 ± 0.206 | 1.01 ± 0.207 |
| | 2 h | 6.21-6.49 | 98.78 ± 0.258 | 1.22 ± 0.257 |

^{a}Time 0 measurements were from a single solution before splitting into triplicates.

### Studies on Lobeline Epimerization in the Buffer Systems Used in the [³H]DTBZ Binding and VMAT2 Uptake Studies.

Lobeline epimerization in deionized water is minimal. However, it could not be assumed that epimerization did not occur in the buffer systems employed in the [³H]DTBZ binding and VMAT2 uptake studies previously described for assessing the mechanism of lobeline activity (Crooks and Dwoskin, U.S. Pat. No. 6,087,376, 2000). Potential epimerization may have had an important role in the pharmacological interactions observed therein, and it was deemed important to assess the epimerization of lobeline in the corresponding buffer systems. In order to determine the tendency of lobeline to undergo epimerization in the HEPES buffer employed in the 25° C. [³H]DTBZ binding study and the 37° C. VMAT2 uptake studies previously described, analogous solution epimerization experiments were conducted using the corresponding conditions for each individual assay. In stark contrast to the results obtained with deionized water, lobeline undergoes rapid epimerization in both buffer systems. As this dynamic phenomenon occurs, the ratio of lobeline to lobeline epimer in solution naturally changes over time. Further, depending upon the solution conditions, the rate and extent of epimerization may vary; thus, a ratio of lobeline to epimer, if achieved, may occur at various time points. After 2 hours, the 25° C. [³H]DTBZ binding buffer system (Table 2) contained 83% starting lobeline and 17% epimer, a rate slower than that observed in a pH 7.4 phosphate buffer control (64% lobeline and 35% epimer, with impurity, after 2 hours). This difference in epimerization at the same pH, concentration and temperature may be attributed to an unknown effect of a HEPES buffer component, thereby underscoring the unpredictable rate and extent of lobeline epimerization in otherwise similar aqueous systems. The epimerization of lobeline in the 37° C. VMAT2 uptake buffer is summarized in Table 3. Here, epimerization was similar in rate and extent to that observed in a phosphate buffer control (62% lobeline and 36% epimer, with impurity, after 2 hours). After 2 hours, the uptake buffer solution contained 69% lobeline, 30% epimer and 1.2% impurity). Reducing the amount of ATP-Mg2+ cofactor in the buffer from 2 mM to 0.4 mM did not substantially alter the rate and extent of lobeline epimerization (66% lobeline, 33% epimer, with impurity, after 2 hours).

Taken together, these data clearly demonstrate that the results obtained in the [³H]DTBZ binding and VMAT2 uptake studies may be attributed to the influence and interaction of a mixture of lobeline and lobeline in solution. This furthers the important consideration that a certain ratio of lobeline and lobeline epimer, or a range of such ratios, rather than the absolute concentration of lobeline, may be critical to the pharmacological and toxicological activity of lobeline in vivo.

TABLE 2
Lobeline conversion to epimer in the buffer system used at 25° C. in the [³H]DTBZ
binding study.

| Time | Sample Age | Impurity B | | Lobeline | | Epimer | |
|---|---|---|---|---|---|---|---|
| Point (min) | (h:min) | RT (min) | Impurity B % | RT (min) | Lobeline % | RT (min) | Epimer % |
| T0 | 0:01 | — | 0.0 | 9.569 | 98.5 | 12.067 | 1.54 |
| T5 | 0:06 | — | 0.0 | 9.565 | 97.8 | 12.042 | 2.16 |
| T10 | 0:10 | — | 0.0 | 9.567 | 96.7 | 12.059 | 3.34 |
| T15 | 0:15 | — | 0.0 | 9.556 | 95.5 | 12.048 | 4.55 |
| T20 | 0:20 | — | 0.0 | 9.556 | 94.7 | 12.044 | 5.27 |
| T25 | 0:25 | — | 0.0 | 9.552 | 94.1 | 12.061 | 5.91 |
| T30 | 0:31 | — | 0.0 | 9.552 | 92.8 | 12.036 | 7.18 |
| T35 | 0:35 | — | 0.0 | 9.556 | 92.7 | 12.036 | 7.30 |
| T40 | 0:40 | — | 0.0 | 9.550 | 91.3 | 12.022 | 8.66 |
| T45 | 0:45 | — | 0.0 | 9.548 | 90.7 | 12.032 | 9.31 |
| T50 | 0:50 | — | 0.0 | 9.548 | 90.1 | 12.024 | 9.94 |
| T55 | 0:55 | — | 0.0 | 9.548 | 89.9 | 12.012 | 10.2 |
| T60 | 1:00 | — | 0.0 | 9.546 | 89.1 | 12.020 | 10.9 |
| T70 | 1:10 | 8.144 | 0.36 | 9.537 | 87.8 | 12.011 | 11.9 |
| T80 | 1:20 | 8.151 | 0.35 | 9.537 | 86.6 | 12.017 | 13.1 |
| T90 | 1:31 | 8.148 | 0.36 | 9.531 | 85.5 | 12.005 | 14.1 |
| T100 | 1:40 | 8.140 | 0.38 | 9.528 | 84.5 | 11.994 | 15.2 |
| T110 | 1:50 | 8.187 | 0.37 | 9.527 | 83.5 | 11.980 | 16.1 |
| T120 | 2:01 | 8.145 | 0.38 | 9.528 | 82.7 | 11.986 | 17.0 |

Sample Age = (Injection Time) − (Time buffer was added)
% Impurity B = [Area Impurity B/(Area Impurity B + Area Lobeline + Area Epimer)] * 100
% Lobeline = [Area Lobeline)/(Area Impurity B + Area Lobeline + Area Epimer)] * 100
% Epimer = [Area Epimer)/(Area Impurity B + Area Lobeline + Area Epimer)] * 100
Note that T0 began one minute after the buffer was added to the aqueous lobeline sulfate solution and placed in the 25° C. constant temperature bath.
Although initial attempts to work at lower concentrations were made, the analytical assay was designed for a test sample concentration of about 0.5 mg/mL rather than the much lower concentrations used in the binding study (1 nM-0.1 mM = 4 × 10⁻⁷ mg/mL-0.04 mg/mL). Under such conditions, the analytical method could not quantitate the early formation of the epimer and impurities due to their low levels.

TABLE 3
Lobeline conversion to epimer in the buffer system used at 37° C. in the VMAT2
uptake study.

| Time | Sample Age | Impurity B | | Lobeline | | Epimer | |
|---|---|---|---|---|---|---|---|
| Point (min) | (h:min) | RT (min) | Impurity B % | RT (min) | Lobeline % | RT (min) | Epimer % |
| T0 | 0:01 | — | 0 | 9.259 | 98.2 | 11.603 | 1.76 |
| T5 | 0:05 | — | 0 | 9.252 | 96.7 | 11.576 | 3.34 |
| T10 | 0:10 | 7.958 | 0.17 | 9.250 | 94.4 | 11.582 | 5.47 |
| T15 | 0:15 | 8.025 | 0.21 | 9.248 | 91.2 | 11.562 | 8.62 |
| T20 | Time point sample lost prior to analysis | | | | | | |
| T25 | 0:25 | 7.952 | 0.32 | 9.243 | 86.5 | 11.549 | 13.2 |
| T30 | 0:30 | 7.977 | 0.39 | 9.239 | 84.7 | 11.550 | 14.9 |
| T35 | 0:35 | 7.994 | 0.42 | 9.238 | 83.4 | 11.546 | 16.2 |
| T40 | 0:40 | 7.978 | 0.53 | 9.235 | 81.8 | 11.539 | 17.7 |
| T45 | 0:45 | 7.940 | 0.49 | 9.230 | 80.6 | 11.530 | 18.9 |
| T50 | 0:50 | 7.972 | 0.57 | 9.232 | 79.3 | 11.523 | 20.1 |
| T55 | 0:55 | 7.959 | 0.50 | 9.227 | 78.5 | 11.523 | 21.0 |
| T60 | 1:00 | 7.977 | 0.66 | 9.225 | 77.2 | 11.516 | 22.1 |
| T70 | 1:10 | 7.976 | 0.71 | 9.223 | 75.0 | 11.511 | 24.3 |
| T80 | 1:20 | 7.955 | 0.85 | 9.218 | 73.1 | 11.504 | 26.1 |
| T90 | 1:31 | 7.923 | 0.83 | 9.215 | 72.2 | 11.497 | 26.9 |
| T100 | 1:40 | 7.945 | 0.92 | 9.212 | 71.1 | 11.493 | 28.0 |
| T110 | 1:50 | 7.954 | 1.16 | 9.209 | 69.6 | 11.486 | 29.2 |
| T120 | 2:00 | 7.954 | 1.16 | 9.204 | 68.5 | 11.478 | 30.3 |

Sample Age = (Injection Time) − (Time buffer was added) % Impurity B = [Area Impurity B/(Area Impurity B + Area Lobeline + Area Epimer)] * 100
% Lobeline = [Area Lobeline)/(Area Impurity B + Area Lobeline + Area Epimer)] * 100
% Epimer = [Area Epimer)/(Area Impurity B + Area Lobeline + Area Epimer)] * 100
Note that T0 begins at the time the buffer is added to the aqueous lobeline sulfate solution and placed in the 37° C. constant temperature bath

In vivo Lobeline Epimerization in Rats.

Table 4 summarizes the in vivo pharmacokinetic data of lobeline and epimer after lobeline administration to male and female rats. Here, serial doses of lobeline were administered to male and female rats intravenously, subcutaneously or orally, and the pharmacokinetic profiles of the parent lobeline and its epimer were monitored in plasma. An examination of the Cmax and areas under the curve (AUC) reveals that significant amounts of the epimer were detected in plasma, irrespective of the dose or route of administration of lobeline, and that significant epimer concentrations were achieved within 15 to 30 min after lobeline administration. A comparison of lobeline and epimer AUC's suggests that the ratio of lobeline to epimer is variable, ranging between ca. 3:2 and 2:1. Further, the rate and extent of epimerization appeared to between sexes, and the systemic absorption of lobeline, followed by subsequent epimerization, was significantly reduced in females as compared to males when given orally. These data suggest that epimerization may take place at different rates in different animals, and that the actual ratio of lobeline to epimer in plasma after lobeline administration is characterized by a fairly broad, and not necessarily predictable, range. Thus, individual variations in epimerization rate and extent may influence the pharmacological activity and therapeutic efficacy of lobeline, perhaps rendering some organisms particularly sensitive to lobeline's therapeutic and toxic effects, and others less so. The failure to take these differences into account may also compromise the development of therapeutically efficacious lobeline regimens.

TABLE 4
Pharmacokinetic parameters of lobeline and epimer after lobeline administration
to rats intravenously (IV), subcutaneously (SC) and orally (PO).

| | | | | | | |
|---|---|---|---|---|---|---|
| Parameters | IV-Female | IV-Male | SC-Female | SC-Male | Oral-Female | Oral-Male |

Lobeline

| | | | | | | |
|---|---|---|---|---|---|---|
| Dose | 2 | 2 | 3 | 3 | 20 | 20 |
| (mg/kg) | | | | | | |
| Tmax (hr.) | 0.083 | 0.083 | 0.25 | 0.083 | 0.5 | 0.5 |
| Cmax | 106 | 144 | 142 | 162 | 42.5 | 91.2 |
| (ng/ml) | | | | | | |
| AUClast | 133.03 | 134.5 | 191.38 | 189.59 | 62.71 | 103.64 |
| (ng * hr/ml) | | | | | | |
| AUCinf | 134.07 | 135.21 | 192.6 | 190.99 | 64.49 | 104.64 |
| (ng * hr/ml) | | | | | | |
| t½ (hr.) | 1.85 | 1.68 | 1.14 | 1.18 | 2.29 | 1.97 |
| MRTlast (hr) | 1.722 | 1.545 | 1.420 | 1.353 | 1.923 | 1.878 |
| Cl | 14.9 | 14.8 | | | | |
| (ml/hr * Kg) | | | | | | |
| Vss | 27.2 | 23.8 | | | | |
| (ml/Kg) | | | | | | |

Lobeline Epimer

| | | | | | | |
|---|---|---|---|---|---|---|
| Dose | 2 | 2 | 3 | 3 | 20 | 20 |
| (mg/kg) | | | | | | |
| Tmax (hr.) | 0.25 | 0.25 | 0.5 | 0.5 | 0.5 | 0.5 |
| Cmax | 53.9 | 47.6 | 85 | 54.4 | 23.8 | 49.8 |
| (ng/ml) | | | | | | |
| AUClast | 75.4 | 68.68 | 126.95 | 108.47 | 39.32 | 53.96 |
| (ng * hr/ml) | | | | | | |
| AUCinf | 76.9 | 69.7 | 127.73 | 109.19 | 40.9 | 54.77 |
| (ng * hr/ml) | | | | | | |
| t½ (hr.) | 1.47 | 1.26 | 1.11 | 1.11 | 1.71 | 1.32 |
| MRTlast (hr) | 1.599 | 1.514 | 1.495 | 1.445 | 2.160 | 1.73 |
| Cl | 26.0 | 28.8 | | | | |
| (ml/hr * Kg) | | | | | | |
| Vss | 45.9 | 46.3 | | | | |
| (ml/Kg) | | | | | | |

In vivo Lobeline Epimerization in Human Volunteers Following Sublingual Lobeline Administration.

The pharmacokinetics of lobeline and lobeline epimer were examined in eight human subjects (Sub) after sublingual lobeline administration. Following sublingual doses of 7.5, 15 and 30 mg lobeline, plasma concentrations of both lobeline and epimer diminished rapidly. Epimer plasma concentrations were approximately 8 to 10-fold greater than those for lobeline, as determined by Cmax and area under the curve analysis. Terminal exponential phase half-lives (T_{1/2} values) for both entities ranged from 1 to 5 hours with an average of approximately 2.4 hours for lobeline and 1.7 hours for epimer. Tₘₐₓ values were in the range of 0.25 to 3 hours with an average of 1.4 and 1.3 hours for lobeline and epimer, respectively, which indicated relatively rapid absorption of the bioavailable sublingual dose. Sublingual clearance and maximum plasma concentration values were consistent with linear pharmacokinetics. Combined urinary excretion of lobeline and epimer never exceeded 0.5%, indicating renal elimination was a minor elimination pathway of these entities. The ratio of the cumulative amount of urinary epimer to lobeline was approximately 0.5, which is the value obtained in vitro with water at pH 7. Plasma concentrations for both lobeline and epimer disappeared relatively rapidly from plasma. Frequently, plasma concentrations could only be quantitated for 2, 4 or 6 hours (nominal times). The mean T_{1/2} values for lobeline and epimer were approximately 2 hours. The mean Tₘₐₓ values for lobeline and its epimer were an average of 1.4 and 1.3 hours for lobeline and epimer, respectively, which indicates fast absorption via the sublingual route. Linear kinetics in Cₘₐₓ and CL/F of lobeline and its epimer were suggested over the dose range from 7.5 to 30 mg. These data suggest that the ratio of epimer to lobeline in plasma after lobeline administration exists at a certain discrete, though variable, range. Notably, these data reveal that the ratio of epimer to the parent lobeline ranges from approximately 8—to approximately 10 to 1, as the parent lobeline is absorbed and distributed in the plasma. This phenomenon is neither obvious nor predictable, based upon the administration of lobeline alone. These observations further underscore the important concept that lobeline may exert its biological effects only when present in certain lobeline to lobeline epimer ratios, and that control of this ratio may be critical to the therapeutic efficacy and potential toxicity of lobeline.

The pharmacokinetic parameters of lobeline after sublingual administration are summarized in Table 5. The pharmacokinetic parameters of lobeline epimer after sublingual administration are summarized in Table 6.

TABLE 5
The pharmacokinetic parameters of lobeline after sublingual administration.

| | | | λz | λz | | | | | | | | | |
|---|---|---|---|---|---|---|---|---|---|---|---|---|---|
| | Wt | Dose | lower | upper | λz | T-½ | Cmax | Tmax | Tlast | AUC(0-t) | AUC(0-∞) | AUCext | CL/F |
| Sub | (kg) | (mg) | (hr) | (hr) | (1/hr) | (hr) | (ng/mL) | (hr) | (hr) | (ng-hr/mL) | (ng-hr/mL) | (%) | (mL/min/kg) |
| 4054 | 72.7 | 7.50 | 2.00 | 4.00 | 0.367 | 1.89 | 0.383 | 1.00 | 4.00 | 1.15 | 1.64 | 29.5 | 1051 |
| 4054 | 72.7 | 15.0 | 3.00 | 6.00 | 0.321 | 2.16 | 0.880 | 2.00 | 6.00 | 2.64 | 3.22 | 18.1 | 1068 |
| 4054 | 72.7 | 30.0 | 4.00 | 8.00 | 0.380 | 1.82 | 1.850 | 2.00 | 8.00 | 7.55 | 8.02 | 5.87 | 857 |
| 4055 | 76.8 | 7.50 | 1.00 | 3.03 | 0.401 | 1.73 | 0.248 | 1.00 | 3.03 | 0.42 | 0.699 | 39.3 | 2328 |
| 4055 | 76.8 | 15.0 | 0.92 | 4.17 | 0.513 | 1.35 | 0.676 | 0.920 | 4.17 | 1.37 | 1.63 | 16.1 | 1995 |
| 4055 | 76.8 | 30.0 | 2.00 | 4.00 | 0.878 | 0.790 | 1.360 | 2.00 | 4.00 | 3.33 | 3.60 | 7.44 | 1809 |
| 4057 | 86.4 | 7.50 | 1.00 | 4.00 | 0.403 | 1.72 | 0.576 | 0.75 | 4.00 | 1.35 | 1.72 | 21.5 | 844 |
| 4057 | 86.4 | 15.0 | 1.00 | 4.00 | 0.544 | 1.27 | 1.27 | 1.00 | 4.00 | 2.87 | 3.34 | 14.1 | 866 |
| 4057 | 86.4 | 30.0 | 2.00 | 6.05 | 0.469 | 1.48 | 2.48 | 0.750 | 6.05 | 4.63 | 4.95 | 6.59 | 1169 |
| 4058 | 63.6 | 7.50 | 3.00 | 4.05 | 0.195 | 3.55 | 0.492 | 1.00 | 4.05 | 1.08 | 1.76 | 38.5 | 1116 |
| 4058 | 63.6 | 15.0 | 4.00 | 6.00 | 0.573 | 1.21 | 1.01 | 1.00 | 6.00 | 2.70 | 2.88 | 6.24 | 1364 |
| 4058 | 63.6 | 30.0 | 4.00 | 8.02 | 0.314 | 2.21 | 2.18 | 0.250 | 8.02 | 5.83 | 6.19 | 5.81 | 1269 |
| 4061 | 90.9 | 7.50 | 3.00 | 6.00 | 0.295 | 2.35 | 0.612 | 2.00 | 6.00 | 1.78 | 2.21 | 19.2 | 623 |
| 4061 | 90.9 | 15.0 | 3.00 | 8.00 | 0.341 | 2.04 | 2.15 | 1.00 | 8.00 | 4.67 | 5.00 | 6.52 | 550 |
| 4061 | 90.9 | 30.0 | 4.00 | 12.17 | 0.254 | 2.73 | 2.49 | 2.00 | 12.17 | 9.13 | 9.62 | 5.16 | 572 |
| 4063 | 77.3 | 7.50 | 3.00 | 6.00 | 0.277 | 2.51 | 0.600 | 1.00 | 6.00 | 1.67 | 2.04 | 18.0 | 792 |
| 4063 | 77.3 | 15.0 | 3.00 | 6.00 | 0.322 | 2.15 | 0.825 | 0.750 | 6.00 | 2.34 | 2.73 | 14.5 | 1183 |
| 4063 | 77.3 | 30.0 | 6.00 | 8.03 | 0.149 | 4.64 | 0.780 | 2.00 | 8.03 | 2.97 | 3.73 | 20.3 | 1736 |
| 4064 | 70.5 | 7.50 | 6.00 | 12.0 | 0.183 | 3.79 | 1.54 | 3.00 | 12.00 | 8.69 | 9.91 | 12.3 | 179 |
| 4064 | 70.5 | 15.0 | 4.00 | 11.0 | 0.206 | 3.36 | 3.36 | 2.00 | 11.00 | 16.48 | 18.87 | 12.7 | 188 |
| 4064 | 70.5 | 30.0 | 6.00 | 22.75 | 0.151 | 4.60 | 6.95 | 3.00 | 22.75 | 42.28 | 43.81 | 3.50 | 162 |
| 4065 | 61.4 | 7.50 | 1.00 | 4.00 | 0.555 | 1.25 | 0.576 | 0.500 | 4.00 | 1.17 | 1.36 | 13.6 | 1502 |
| 4065 | 61.4 | 15.0 | 3.00 | 8.00 | 0.185 | 3.75 | 1.03 | 1.00 | 8.00 | 2.77 | 3.41 | 18.7 | 1194 |
| 4065 | 61.4 | 30.0 | 4.00 | 6.00 | 0.376 | 1.84 | 1.40 | 1.00 | 6.00 | 3.17 | 3.48 | 8.86 | 2340 |

NA = not applicable;
ND = not determinable;
λz = terminal exponential rate constant;
λz (lower) and λz (upper) are the first and last data points used for calculation of λz, respectively;
T-½ = terminal exponential half-life;
Cmax = maximum plasma concentration;
Tmax = time of Cmax; AUC(0-t) = area under the plasma concentration-time curve from time 0 to the last measurable data point;
Tlast = time of last measurable plasma concentration;
AUC(0-∞) = area under the plasma concentration-time curve from time 0 to infinity;
AUCext (%) = percent of AUC(0-∞) determined by extrapolation;
CL = clearance;
F = absolute bioavailability; and
CL/F = extravascular clearance.

TABLE 6
The pharmacokinetic parameters of epimer after sublingual administration.

| | | | | | | | | | | | | | | AUC(0-∞) |
|---|---|---|---|---|---|---|---|---|---|---|---|---|---|---|
| | Wt | Dose | λz lower | λz upper | λz | T-½ | Cmax | Tmax | Tlast | AUC(0-t) | AUC(0-∞) | AUCext | CL/F | Ratio |
| Sub | (kg) | (mg) | (hr) | (hr) | (1/hr) | (hr) | (ng/mL) | (hr) | (hr) | (ng-hr/mL) | (ng-hr/mL) | (%) | (mL/min/kg) | (Epi/Lob) |
| 4054 | 72.7 | 7.50 | 2.00 | 4.00 | 0.489 | 1.418 | 3.43 | 2.00 | 4.00 | 9.28 | 11.92 | 22.14 | 144.2 | 7.29 |
| 4054 | 72.7 | 15.0 | 3.00 | 6.00 | 0.5175 | 1.339 | 9.22 | 1.00 | 6.00 | 32.47 | 35.33 | 8.10 | 97.3 | 10.97 |
| 4054 | 72.7 | 30.0 | 4.00 | 8.00 | 0.3741 | 1.853 | 24.6 | 2.00 | 8.00 | 87.17 | 91.76 | 5.01 | 74.9 | 11.44 |
| 4055 | 76.8 | 7.50 | 1.00 | 2.00 | 0.6868 | 1.009 | 3.16 | 1.00 | 2.00 | 3.44 | 5.76 | 40.22 | 282.7 | 8.23 |
| 4055 | 76.8 | 15.0 | 2.17 | 4.17 | 0.6055 | 1.145 | 8.48 | 0.920 | 4.17 | 15.44 | 17.29 | 10.70 | 188.2 | 10.60 |
| 4055 | 76.8 | 30.0 | 3.00 | 6.00 | 0.6184 | 1.121 | 16.4 | 1.00 | 6.00 | 40.70 | 42.35 | 3.89 | 153.7 | 11.77 |
| 4057 | 86.4 | 7.50 | 1.00 | 3.00 | 0.3858 | 1.797 | 4.42 | 0.500 | 3.00 | 8.22 | 13.15 | 37.47 | 110.1 | 7.66 |
| 4057 | 86.4 | 15.0 | 2.00 | 4.00 | 0.6196 | 1.119 | 8.71 | 1.00 | 4.00 | 20.90 | 23.97 | 12.80 | 120.8 | 7.17 |
| 4057 | 86.4 | 30.0 | 2.00 | 4.00 | 0.6931 | 1.000 | 14.6 | 0.750 | 4.00 | 28.10 | 31.37 | 10.44 | 184.5 | 6.33 |
| 4058 | 63.6 | 7.50 | 1.00 | 3.00 | 0.6119 | 1.133 | 3.57 | 1.00 | 3.00 | 6.45 | 8.16 | 21.02 | 240.7 | 4.64 |
| 4058 | 63.6 | 15.0 | 2.00 | 4.00 | 0.4338 | 1.598 | 9.22 | 1.00 | 4.00 | 18.86 | 24.30 | 22.39 | 161.7 | 8.44 |
| 4058 | 63.6 | 30.0 | 2.00 | 6.00 | 0.5281 | 1.313 | 20.0 | 0.750 | 6.00 | 50.50 | 53.47 | 5.56 | 146.9 | 8.63 |
| 4061 | 90.9 | 7.50 | 3.00 | 6.00 | 0.3882 | 1.785 | 6.07 | 2.00 | 6.00 | 16.86 | 19.46 | 13.37 | 70.7 | 8.82 |
| 4061 | 90.9 | 15.0 | 3.00 | 8.00 | 0.3591 | 1.930 | 21.8 | 1.00 | 8.00 | 46.10 | 48.91 | 5.75 | 56.2 | 9.79 |
| 4061 | 90.9 | 30.0 | 3.00 | 8.00 | 0.25 | 2.772 | 25.6 | 2.00 | 8.00 | 83.89 | 99.13 | 15.37 | 55.5 | 10.30 |
| 4063 | 77.3 | 7.50 | 2.00 | 3.00 | 0.531 | 1.305 | 3.58 | 1.00 | 3.00 | 7.20 | 10.16 | 29.10 | 159.2 | 4.97 |
| 4063 | 77.3 | 15.0 | 2.00 | 4.00 | 0.684 | 1.013 | 6.81 | 1.00 | 4.00 | 16.40 | 18.42 | 10.95 | 175.6 | 6.74 |
| 4063 | 77.3 | 30.0 | 2.00 | 4.00 | 0.7194 | 0.964 | 7.63 | 2.00 | 4.00 | 20.12 | 22.64 | 11.12 | 285.9 | 6.07 |
| 4064 | 70.5 | 7.50 | 4.00 | 12.00 | 0.2004 | 3.459 | 15.6 | 2.00 | 12.00 | 90.70 | 101.93 | 11.02 | 17.4 | 10.29 |
| 4064 | 70.5 | 15.0 | 3.00 | 11.00 | 0.1908 | 3.633 | 45.0 | 2.00 | 11.00 | 208.40 | 244.24 | 14.68 | 14.5 | 12.94 |
| 4064 | 70.5 | 30.0 | 6.00 | 22.75 | 0.1513 | 4.582 | 82.0 | 3.00 | 22.75 | 501.67 | 518.59 | 3.26 | 13.7 | 11.84 |
| 4065 | 61.4 | 7.50 | 1.00 | 3.00 | 0.6101 | 1.136 | 4.59 | 0.500 | 3.00 | 7.13 | 9.03 | 21.05 | 225.5 | 6.66 |
| 4065 | 61.4 | 15.0 | 2.00 | 4.03 | 0.7266 | 0.954 | 10.5 | 1.00 | 4.03 | 20.66 | 22.61 | 8.64 | 180.2 | 6.63 |
| 4065 | 61.4 | 30.0 | 2.00 | 4.00 | 0.8194 | 0.846 | 16.4 | 1.00 | 4.00 | 28.95 | 31.41 | 7.85 | 259.4 | 9.02 |

Epi = Epimer;
Lob = lobeline;
NA = not applicable;
ND = not determinable;
λz = terminal exponential rate constant;
λz (lower) and λz (upper) are the first and last data points used for calculation of λz, respectively;
T-½ = terminal exponential half-life;
Cmax = maximum plasma concentration;
Tmax = time of Cmax;
AUC(0-t) = area under the plasma concentration-time curve from time 0 to the last measurable data point;
Tlast = time of last measurable plasma concentration;
AUC(0-∞) = area under the plasma concentration-time curve from time 0 to infinity;
AUCext (%) = percent of AUC(0-∞) determined by extrapolation;
CL = clearance;
F = absolute bioavailability; and
CL/F = extravascular clearance.

### Pharmacokinetics of Lobeline and Epimer After Three Ascending and Repeated Doses (Up to 60 mg) of Sublingual Lobeline in Healthy Volunteers.

Three consecutive doses of lobeline (either 30, 45 and 45 mg in the first study group, or 60, 60 and 60 mg in the second group) were administered to human volunteers at 0800, 1200 and 1600 over two full inpatient days. The pharmacokinetics of lobeline and epimer were then assessed in each subject. Visual inspection of plasma concentration-time curves revealed that there was a high degree of variability for both lobeline and epimer between subjects. However, the epimer to lobeline plasma concentration ratios maintained a consistent value near 10. At each time point, the percent CV values were relatively low, near 30%. Consequently, higher lobeline concentrations were associated with proportionally higher epimer concentrations throughout. A crude index of accumulation (non-steady state) was obtained by dividing the sixth dose trough concentration by that observed for the fifth dose. These ratios were in the vicinity of 1.2 to 1.3. Following the last dose, the geometric mean lobeline plasma concentration peaked at 1.26 hours, whereas the corresponding epimer mean plasma concentration peaked at 1.38 hours. The geometric mean values for the half lives of lobeline and epimer were 3.91 and 3.47 hours, respectively.

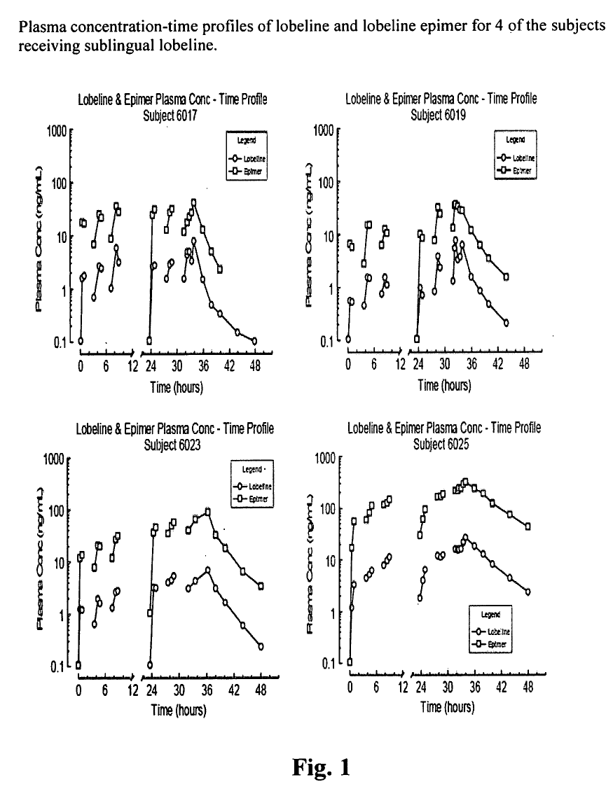

FIG. 1 depicts plasma concentration-time curves for 4 of the subjects. Once again, this phenomenon is neither obvious nor predictable. Again, these observations further suggest that in vivo epimer to lobeline ratios are dynamic, that lobeline may exert its biological effects only when present in certain lobeline to epimer ratios, and that control of this ratio may be critical to the therapeutic efficacy and potential toxicity of lobeline.

These data indicate that the intersubject and interspecies variability of lobeline and epimer pharmacokinetics is high, and not necessarily predictable. Further, although the overall ratio of epimer to lobeline in plasma is approximately 10:1, this ratio may change over time, and may be influenced by the dose of lobeline administered. The dynamic and variable nature of lobeline epimerization in the plasma is once again demonstrated. Since lobeline is known to undergo epimerization in the pharmacological assays previously described and, consequently, that the ratio of lobeline to epimer changes over time during these studies, it is clear that the lobeline to epimer ratio may profoundly impact lobeline pharmacology. These various in vivo studies reveal that lobeline epimerization also occurs in a dynamic and unpredictable manner in humans and other mammals. It is logical to infer that a specific lobeline to epimer ratio, or a dynamic range of lobeline to epimer ratios, may be necessary to achieve the desired pharmacological effect of lobeline in mammals. Thus, another aspect of theis invention is to teach that the administration, delivery or achievement of a specific lobeline to epimer ratio or a range of ratios is most desirable when using lobeline therapeutically.

The description of the embodiments of the present invention is given above for the understanding of the present invention. It will be understood that the invention is not limited to the particular embodiments described herein, but is capable of various modifications, rearrangements and substitutions as will now become apparent to those skilled in the art without departing from the scope of the invention. Therefore, it is intended that the following claims cover all such modifications and changes as fall within the true spirit and scope of the invention.

We claim:

1. A method of treatment of a disease or pathology in a patient in need thereof, comprising:
delivering to the patient an effective amount of an epimeric mixture of 2-[6S-(2S-hydroxy-2-phenyl-ethyl)-1-methyl-piperidin-2R-yl]-1-phenyl-ethanone (2R-lobeline) and its epimer, 2-[6S-(2S-hydroxy-2-phenyl-ethyl)-1-methyl-piperidin-2S-yl]-1-phenyl-ethanone (2S-lobeline), wherein 2R-lobeline and 2S-lobeline have the following structural formulas:

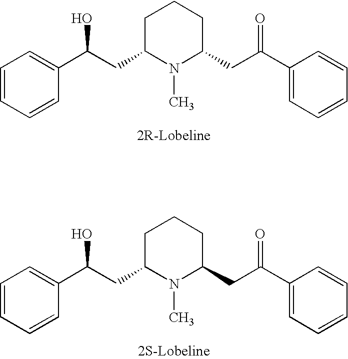

2. The method of claim 1, wherein the disease or pathology is of the central nervous system and delivering the effective dose blocks monoamine and indoleamine uptake into presynaptic terminals or vesicles.

3. The method of claim 1, wherein the disease or pathology is a dependence on a drug of abuse, comprising withdrawal from the drug of abuse or for reducing the patient's desire for food.

4. The method of claim 1, wherein said delivery is achieved by administering the mixture subcutaneously, intramuscularly, intravenously, intrathecally, transdermally, orally, intranasally, sublingually, by inhalation or insufflation, by implantation, or rectally.

5. The method of claim 2, wherein the blockage of monoamine and indoleamine uptake occurs at neuronal nicotinic acetylcholine receptors.

6. The method according to claim 2, wherein said monoamine and indoleamine are dopamine transporter (DAT), norepinephrine transporter (NET), and serotonin transporter (SERT).

7. The method of claim 2, wherein the blockage of monoamine uptake occurs at a vesicular monoamine transporter.

8. The method according to claim 7, wherein said vesicular monoamine transporter is VMAT2.

9. The method of claim 2, wherein the blockage of monoamine uptake occurs at presynaptic monoamine and indoleamine transporters.

10. The method of claim 2, wherein the central nervous system disease or pathology is selected from the group consisting of head or brain trauma, pain management, psychosis, affective disorders, personality disorders, sleep disorders, eating disorders including obesity, obsessive-compulsive disorders, panic disorders, schizophrenia, myasthenia gravis, Parkinson's disease, hyperkinetic disorders, Tourette's syndrome, Huntington's disease, and attention deficit hyperactivity, conduct disorders and drug abuse; wherein said drug of abuse is selected from the group consisting of cocaine, amphetamines, caffeine, phencyclidine, opiates, barbiturates, benzodiazepines, cannabinoids, hallucinogens, psychedelics, and alcohol.

11. The method of claim 1, wherein the epimer mixture, (2R/2S-lobeline), delivered systemically, comprises a mixture of 2R-lobeline and 2S-lobeline in any epimeric ratio of 2R- and 2S-lobeline, ranging between 1 part 2R-lobeline to 10000 parts 2S-lobeline and 10000 parts 2R-lobeline to 1 part 2S-lobeline, or is a 1 to 1 mixture of 2R- to 2S-lobeline.

12. The method of claim 1, wherein the epimer mixture, (2R/2S-lobeline), delivered systemically, produces an epimeric ratio, in the plasma of a mammal so treated, where said plasma ratio of 2R- and 2S-lobeline ranges between 1 part 2R-lobeline to 100 parts 2S-lobeline and 100 parts 2R-lobeline to 1 part 2S-lobeline, or is a 1 to 1 mixture of 2R- to 2S-lobeline.

13. The method of claim 1, wherein the epimer mixture, (2R/2S-lobeline), delivered systemically, produces an epimeric ratio, in the plasma of a mammal so treated, where said plasma ratio of 2R- and 2S-lobeline ranges between 1 part 2R-lobeline to 30 parts 2S-lobeline and 30 parts 2R-lobeline to 1 part 2S-lobeline, or is a 1 to 1 mixture of 2R- to 2S-lobeline.

14. The method of claim 1, wherein the epimer mixture, (2R/2S-lobeline), delivered systemically, produces an epimeric ratio, in the plasma of a mammal so treated, where said plasma ratio of 2R- and 2S-lobeline ranges between 1 part 2R-lobeline to 10 parts 2S-lobeline and 10 parts 2R-lobeline to 1 part 2S-lobeline, or is a 1 to 1 mixture of 2R- to 2S-lobeline.

15. The method of claim 1, wherein said epimer mixture is administered as the free base or as pharmaceutically acceptable salts, solvates, complexes, dispersions or polymorphs thereof

16. The method of claim 3, wherein the patient's desire for said food is reduced for at least one day.

17. The method of claim 3, wherein patient's desire for said drug of abuse is reduced for at least one day.

18. The method of claim 1, which further comprises co-administration of behavior modification counseling to the patient.

19. An in vivo stabilized 2R- and 2S-Lobeline composition in blood, plasma, tissue or cytosol, comprising:
a predetermined epimer mixture ranging between 1 part 2R-lobeline to 10000 parts 2S-lobeline and 10000 parts 2R-lobeline to 1 part 2S-lobeline, or a 1 to 1 mixture of 2R- and 2S-lobeline, wherein 2R-lobeline and 2S-lobeline have the following structural formulas: 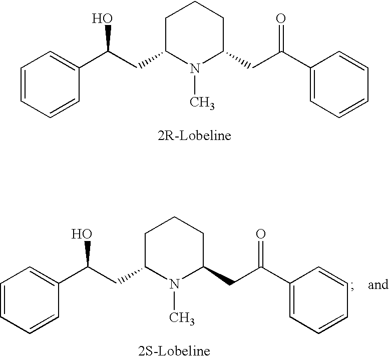
a solvent or one or more pharmaceutical excipients.

20. The composition of claim 19, wherein the solvent is selected from the group consisting of water, saline, aqueous buffers, acetone, ethanol, methanol, isopropanol, isobutanol, tertiary butanol, ethyl acetate, methylene chloride, acetonitrile, glycerin, propylene glycol, liquid paraffin, mineral oil, ethylene glycol, butanol, ethoxyethanol, ethyl ether, isobutyl acetate, isopropyl acetate, propanol, chloroform, butyl acetate, diethylene glycol monoethyl ether, dimethyl sulfoxide, methane sulfonyl methane, and combinations thereof, and their polymorphs.

22. The composition of claim 19, wherein the pharmaceutical excipient is selected from the group consisting of solvents, such as ethanol and diethylene glycol monoethyl ether, surfactants, such as polysorbates, lecithin, fatty acid salts and alcohols, polymers, adhesives such as acrylates and polycarboxylates, binders, fillers and bulking agents, such as starch, lactose and mannitol, preservatives, such as tocopherol, and BHT, and combinations thereof.

23. A method for delivering a stabilized epimeric mixture of 2R- and 2S-lobeline having a predetermined epimeric ratio, comprising:
providing the epimeric mixture having a ratio of 2R- and 2S-lobeline ranging between 1 part 2R-lobeline to 10000 parts 2S-lobeline and 10000 parts 2R-lobeline to 1 part 2S-lobeline, or a 1 to 1 mixture of 2R- and 2S-lobeline, so that it is effective for delivering the predetermined epimeric mixture to the blood, plasma or tissues of an individual and a solvent or one or more pharmaceutical excipients.

24. The method of claim 20, wherein the solvent is selected from the group consisting of water, saline, aqueous buffers, acetone, ethanol, methanol, isopropanol, isobutanol, tertiary butanol, ethyl acetate, methylene chloride, acetonitrile, glycerin, propylene glycol, liquid paraffin, mineral oil, ethylene glycol, butanol, ethoxyethanol, ethyl ether, isobutyl acetate, isopropyl acetate, propanol, chloroform, butyl acetate, diethylene glycol monoethyl ether, dimethyl sulfoxide, methane sulfonyl methane, and combinations thereof, and their polymorphs.

25. The method of claim 20, wherein the pharmaceutical excipient is selected from the group consisting of solvents, such as ethanol and diethylene glycol monoethyl ether, surfactants, such as polysorbates, lecithin, fatty acid salts and alcohols, polymers, adhesives such as acrylates and polycarboxylates, binders, fillers and bulking agents, such as starch, lactose and mannitol, preservatives, such as tocopherol, and BHT, and combinations thereof.
